(19) United States
(12) Patent Application Publication
(10) Pub. No.: US 2026/0165696 A1
(43) Pub. Date: Jun. 18, 2026

(54) LEFT ATRIAL APPENDAGE CLOSURE DEVICES

(71) Applicant: BOSTON SCIENTIFIC SCIMED, INC., MAPLE GROVE, MN (US)

(72) Inventors: Troy Anthony Giese, Blaine, MN (US); Philip Andrew Litecky, Forest Lake, MN (US)

(73) Assignee: BOSTON SCIENTIFIC SCIMED, INC., MAPLE GROVE, MN (US)

(21) Appl. No.: 19/423,171

(22) Filed: Dec. 17, 2025

Related U.S. Application Data
(60) Provisional application No. 63/735,537, filed on Dec. 18, 2024.

(51) Int. Cl. A61B 17/00

(52) U.S. Cl. CPC A61B 17/0057; A61B 2017/00367; A61B 2017/0061; A61B 2017/00632; A61B 2017/00867

(57) ABSTRACT

A left atrial appendage closure (LAAC) device includes an expandable anchor frame and an expandable foam body. The anchor frame expands from a delivery configuration to an expanded anchor configuration, while the foam body expands from a compressed configuration to an expanded configuration. The expansion of these components is temporally offset from each other. The device may include an intermediate structure joining the anchor frame to the foam body. The anchor frame can be actuated through translation or rotation relative to other components. In some embodiments, the foam body expands before the anchor frame. The anchor frame may include struts extending between a threaded distal hub and a secured proximal hub, with expansion achieved through rotation. The device enables controlled deployment for occluding a patient's left atrial appendage.

### CROSS-REFERENCE TO RELATED APPLICATIONS

This application claims the benefit of priority of U.S. Provisional Application No. 63/735,537 filed Dec. 18, 2024, the entire disclosure of which is hereby incorporated by reference.

### TECHNICAL FIELD

The disclosure relates generally to medical devices and more particularly to medical devices that incorporate a shape memory foam component.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding a patient's left atrial appendage (LAA). The LAAC device includes an expandable anchor frame that is adapted to expand from a delivery configuration to an expanded anchor configuration, and an expandable foam body that is disposed relative to the expandable anchor frame and that may be adapted to expand from a compressed configuration to an expanded configuration. Expansion of the expandable anchor frame may be temporally offset from expansion of the expandable foam body.

Alternatively or additionally, the expandable anchor frame may be adapted to expand before the expandable foam body expands.

Alternatively or additionally, the expandable foam body may be adapted to expand before the expandable anchor frame expands.

Alternatively or additionally, the expandable anchor frame may be adapted to move between the delivery configuration and the expanded anchor configuration by translating the expandable anchor frame relative to the expandable foam body.

Alternatively or additionally, the device may include a channel extending axially through the expandable foam body.

Alternatively or additionally, the expandable anchor frame may include a plurality of individually actuatable struts slidingly disposed within the channel.

Alternatively or additionally, each of the plurality of individually actuatable struts may be securable relative to the LAAC device.

Alternatively or additionally, the device may include an intermediate structure joining the expandable anchor frame to the expandable foam body.

Alternatively or additionally, the expandable anchor frame may be adapted to move between the delivery configuration and the expanded anchor configuration by rotating the expandable anchor frame relative to the intermediate structure.

Alternatively or additionally, the expandable frame may include a plurality of struts extending between a distal hub threadedly engaged with the intermediate structure and a proximal hub secured to the intermediate structure.

Alternatively or additionally, the expandable anchor frame may be adapted to move between the delivery configuration and the expanded anchor configuration by rotating the intermediate structure relative to the distal hub.

Another example may be found in a left atrial appendage closure (LAAC) device that is adapted for occluding a patient's left atrial appendage (LAA). The LAAC device includes an expandable anchor frame that is adapted to expand from a delivery configuration to an expanded anchor configuration, and an expandable foam body that is disposed relative to the expandable anchor frame and is adapted to expand from a compressed configuration to an expanded configuration before the expandable anchor frame is expanded.

Alternatively or additionally, the expandable anchor frame may be adapted to translate relative to the expandable foam body.

Alternatively or additionally, the device may include an intermediate structure joining the expandable anchor frame to the expandable foam body.

Alternatively or additionally, the expandable anchor frame may be adapted to move between the delivery configuration and the expanded anchor configuration by rotating the expandable anchor frame relative to the intermediate structure.

Alternatively or additionally, the expandable frame may comprise a plurality of struts extending between a distal hub threadedly engaged with the intermediate structure and a proximal hub secured to the intermediate structure.

Alternatively or additionally, the expandable anchor frame may be adapted to move between the delivery configuration and the expanded anchor configuration by rotating the intermediate structure relative to the distal hub.

Another example may be found in a left atrial appendage closure (LAAC) device that is adapted for occluding a patient's left atrial appendage (LAA). The LAAC device includes an expandable anchor frame that is adapted to expand from a delivery configuration to an expanded anchor configuration, an expandable foam body disposed relative to the expandable anchor frame that is adapted to expand from a compressed configuration to an expanded configuration, and an intermediate structure that joins the expandable anchor frame to the expandable foam body.

Alternatively or additionally, the expandable anchor frame may be adapted to move between the delivery configuration and the expanded anchor configuration by rotating the expandable anchor frame relative to the intermediate structure.

Alternatively or additionally, the expandable frame may include a plurality of struts that extend between a distal hub that is threadedly engaged with the intermediate structure and a proximal hub that is secured to the intermediate structure. The expandable anchor frame may be adapted to move between the delivery configuration and the expanded anchor configuration by rotating the intermediate structure relative to the distal hub.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a schematic view of an illustrative LAAC device shown in the LAA, after expansion of an expandable foam body and before expansion of an expandable anchor frame;

FIG. 3 is a schematic view of the illustrative LAAC device of FIG. 2, shown after expansion of the expandable anchor frame;

FIG. 4 is a schematic view of an illustrative LAAC device shown in the LAA, after expansion of an expandable foam body and before expansion of an expandable anchor frame;

FIG. 5 is a schematic view of the illustrative LAAC device of FIG. 4, shown after expansion of the expandable anchor frame;

FIG. 6 is a schematic view of an illustrative LAAC device shown in the LAA, after expansion of an expandable foam body and before expansion of an expandable anchor frame;

FIG. 7 is a schematic view of the illustrative LAAC device of FIG. 6, shown after expansion of the expandable anchor frame;

FIG. 8 is a schematic view of an illustrative LAAC device shown in the LAA, after expansion of an expandable foam body and before expansion of an expandable anchor frame;

FIG. 9 is a schematic view of the illustrative LAAC device of FIG. 8, shown after expansion of the expandable anchor frame;

FIG. 10 is a schematic view of an illustrative LAAC device shown in the LAA, after expansion of an expandable foam body and before expansion of an expandable anchor frame;

FIG. 11 is a schematic view of the illustrative LAAC device of FIG. 10, shown after expansion of the expandable anchor frame;

FIG. 12 is a perspective view of the expandable anchor frame forming part of the illustrative LAAC device of FIG. 10, shown in an expanded configuration;

FIG. 12A is an enlarged view of a portion of the expandable anchor frame shown in FIG. 12;

FIG. 13 is a schematic view of an illustrative LAAC device, shown after expansion of an expandable foam body and before expansion of an expandable anchor frame;

FIG. 14 is a schematic view of the illustrative LAAC device of FIG. 13, shown after expansion of the expandable anchor frame; and

FIG. 15 is a perspective view of a portion of an illustrative LAAC device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic. However, every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

In some instances, a left atrial appendage closure (LAAC) device may be adapted for occluding a patient's left atrial appendage (LAA). The LAAC device includes an expandable anchor frame that can expand from a delivery configuration to an expanded anchor configuration, and an expandable foam body that is disposed relative to the expandable anchor frame. The expandable foam body can expand from a compressed configuration to an expanded configuration. The expansion of these components is temporally offset from one another, meaning that one of the components expands before the other component expands.

In some cases, the expandable anchor frame may expand before the expandable foam body expands. In some cases, the expandable foam body may expand before the expandable anchor frame expands. In some cases, expansion of the expandable anchor frame may be instantaneous or nearly instantaneous while expansion of the expandable foam body may take up to twenty minutes or longer. In some cases, the expandable anchor frame may move between configurations by translating relative to the expandable foam body. In some cases, the device may include a channel extending axially through the expandable foam body. In some cases, the expandable anchor frame may include multiple individually actuatable anchor segments slidingly disposed within the channel. In some cases, each of the individually actuatable anchor segments may be secured relative to the LAAC device. In some cases, the device may include an intermediate structure joining the expandable anchor frame to the expandable foam body. In some cases, the expandable anchor frame may move between configurations by rotating relative to the intermediate structure. In some cases, the expandable anchor frame may include struts extending between a distal hub threadedly engaged with the intermediate structure and a proximal hub secured to the intermediate structure. In some cases, the expandable anchor frame may move between configurations by rotating the intermediate structure relative to the distal hub.

In some instances, an LAAC device may include an expandable anchor frame and an expandable foam body. The expandable foam body expands from a compressed configuration to an expanded configuration before the expandable anchor frame expands.

In some cases, the expandable anchor frame may translate relative to the expandable foam body. In some cases, the device may include an intermediate structure joining the expandable anchor frame to the expandable foam body. In some cases, the expandable anchor frame may move between configurations by rotating relative to the intermediate structure. In some cases, the expandable frame may include struts extending between a distal hub threadedly engaged with the intermediate structure and a proximal hub secured to the intermediate structure. In some cases, the expandable anchor frame may move between configurations by rotating the intermediate structure relative to the distal hub.

In some instances, an LAAC device may include an expandable anchor frame, an expandable foam body, and an intermediate structure joining the anchor frame to the foam body. In some cases, the expandable anchor frame may move between configurations by rotating relative to the intermediate structure. In some cases, the expandable frame may include struts between a threaded distal hub and secured proximal hub. The anchor frame may move between configurations by rotating the intermediate structure relative to the distal hub.

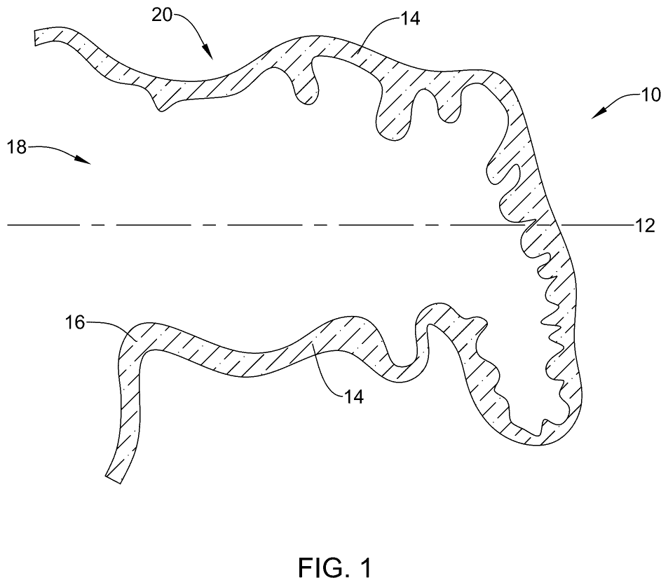

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12. It will be appreciated that FIG. 1 shows an LAA 10 that is just one example of what a left atrial appendage may look like.

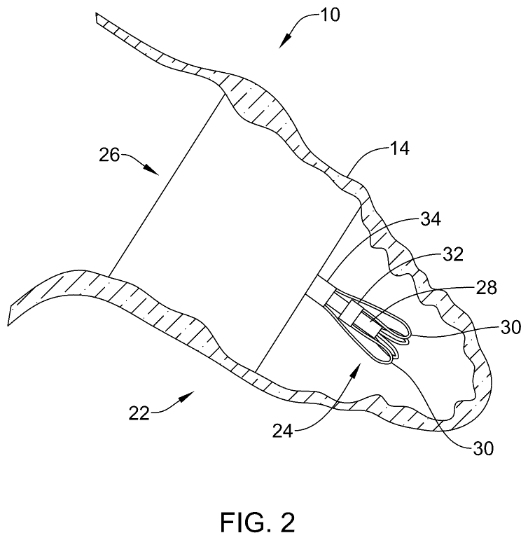

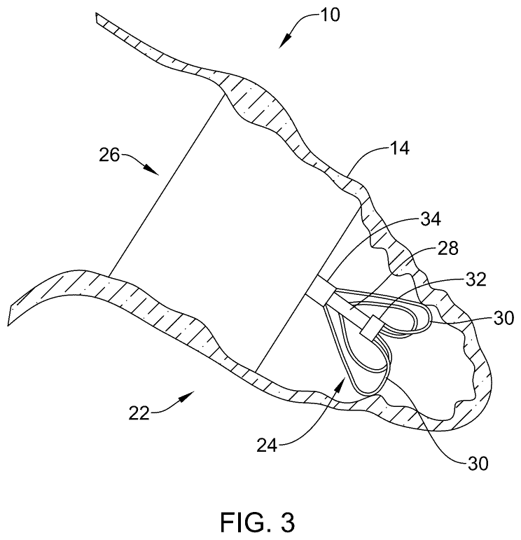

FIG. 2 is a schematic view of an illustrative LAAC device 22 shown disposed within the LAA 10 and FIG. 3 is a schematic view of the illustrative LAAC device 22 of FIG. 2, shown after expansion of an expandable anchor frame. While not shown, at this stage the LAAC device 22 may be coupled with a delivery device. The illustrative LAAC device 22 includes an expandable anchor frame 24 that is expandable from a delivery configuration as shown in FIG. 2 to an expanded anchor configuration as shown for example in FIG. 3. The LAAC device 22 includes an expandable foam body 26 that is expandable from a compressed configuration (not shown) to an expanded configuration as shown in FIGS. 2 and 3. In some cases, the expandable foam body 26 may be adapted to expand from its compressed configuration to its expanded configuration as soon as the expandable foam body 26 is released from a constraining sleeve that holds the expandable foam body 26 in its compressed configuration. In some cases, the expandable foam body 26 may be adapted to regain a remembered shape once no longer constrained, or once the expandable foam body 26 is exposed to elevated temperature and/or moisture (e.g., contacts blood).

In some cases, as shown, the LAAC device 22 also includes an intermediate structure 28. In some cases, the intermediate structure 28 may play a role in moving the expandable anchor frame 24 between its delivery configuration to its expanded anchor configuration. In some cases, the intermediate structure 28 may extend through the expandable foam body 26 such that a delivery device (not shown) may be used to engage the intermediate structure 28 in a manner that allows actuation of the expandable anchor frame 24. The expandable anchor frame 24 may include a plurality of struts 30 that extend between a distal hub 32 and a proximal hub 34. The struts 30 may be made of a shape memory material such as nitinol. The distal hub 32 may be threadedly engaged with a corresponding threaded outer surface on the intermediate structure 28 such that rotation of the intermediate structure 28 relative to the distal hub 32 causes the distal hub 32 to translate along the intermediate structure 28. In some cases, the proximal hub 34 is adapted to allow the intermediate structure 28 to rotate relative to the proximal hub 34 without threadedly engaging the intermediate structure 28. In some cases, the proximal hub 34 may be secured to or within the expandable foam body 26. The intermediate structure 28 may extend proximally through the expandable foam body 26 so that a delivery device (not shown) is able to reach the intermediate structure 28 in order to rotate the intermediate structure 28. In some cases, the delivery device may instead extend through the expandable foam body 26 in order to engage the intermediate structure 28.

In FIG. 2, the distal hub 32 is positioned relatively close to the proximal hub 34, with each of the struts 30 doubled over on themselves in the delivery configuration. The delivery configuration may be considered as representing a compressed or collapsed configuration. By moving the distal hub 32 in a distal direction, away from the proximal hub 34, each of the struts 30 are caused to move in a radially outward direction. As the struts 30 move in a radially outward direction, as shown in FIG. 3, the struts 30 are caused to engage tissue within the LAA 10, thereby helping to anchor the LAAC device 22 in position within the LAA 10. The anchoring provided by the expandable anchor frame 24, along with the anchoring provided by the expandable foam body 26 in its expanded configuration, together secure the LAAC device 22 in position. The combination of the intermediate structure 28 and the distal hub 32 that is threadedly engaged with the intermediate structure 28 allows for the physician implanting the LAAC device 22 to customize the relative spread of the struts 30 to work with a particular patient and their particular left atrial appendage anatomy. The expandable anchor frame 24 may include two, three, four, five, or six struts 30. A maximum number of struts 30 may be determined by overall compressibility of the expandable anchor frame 24 and the capabilities of the delivery device. While in an expanded configuration, the struts 30 may extend radially outwardly from the expandable anchor frame 24.

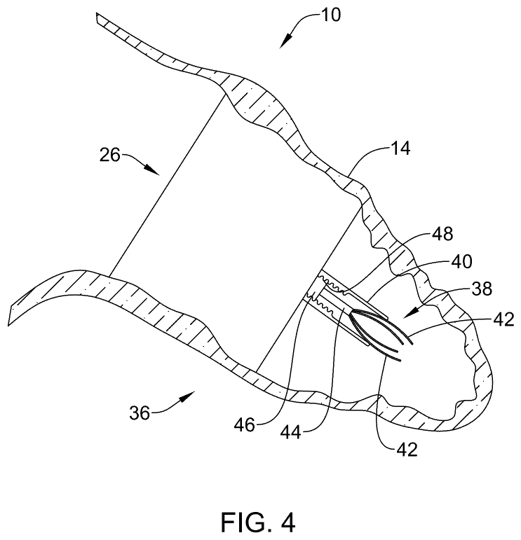

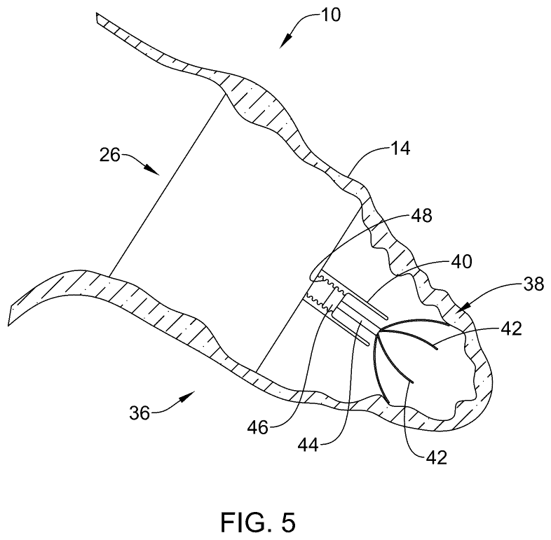

FIG. 4 is a schematic view of an illustrative LAAC device 36 shown disposed within the LAA 10 and FIG. 5 is a schematic view of the illustrative LAAC device 36 of FIG. 4, shown after expansion of an expandable anchor frame. While not shown, at this stage the LAAC device 36 may be coupled with a delivery device. The illustrative LAAC device 36 includes an expandable anchor frame 38 that is expandable from a delivery configuration as shown in FIG. 4 to an expanded anchor configuration as shown for example in FIG. 5. The LAAC device 36 includes an expandable foam body 26 that is expandable from a compressed configuration (not shown) to an expanded configuration as shown in FIGS. 4 and 5. In some cases, the expandable foam body 26 may be adapted to expand from its compressed configuration to its expanded configuration as soon as the expandable foam body 26 is released from a constraining sleeve that holds the expandable foam body 26 in its compressed configuration. In some cases, the expandable foam body 26 may be adapted to regain a remembered shape once no longer constrained, or once the expandable foam body 26 is exposed to increases in temperature and/or moisture (e.g., contacts blood).

In some cases, as shown, the LAAC device 36 also includes an intermediate structure 40. In some cases, the intermediate structure 40 may play a role in moving the expandable anchor frame 38 between its delivery configuration to its expanded anchor configuration. In some cases, the intermediate structure 40 may extend through the expandable foam body 26 such that a delivery device (not shown) may be used to engage the intermediate structure 40 in a manner that allows actuation of the expandable anchor frame 38. The expandable anchor frame 38 may include a plurality of struts 42 that extend distally from a central member 44. The struts 42 may be made of a shape memory material such as nitinol. In some cases, the central member 44 includes a threaded region 46 that is adapted to threadedly engage a threaded region 48 of the intermediate structure 44. Relative rotation of the threaded region 46 relative to the intermediate structure 44 may cause the threaded region 46 of the central member 44 to move distally relative to the intermediate structure 44. As a result, the struts 42 are no longer constrained by the intermediate structure 44, but are able to move in a radially outwardly direction, as seen for example in FIG. 5. In FIG. 5, the expandable anchor frame 38 has moved into its expanded configuration in which the struts 42 are engaging the wall 14 of the LAA 10. The expandable anchor frame 38 may include two, three, four, five, or six struts 42. A maximum number of struts 42 may be determined by overall compressibility of the expandable anchor frame 38 and the capabilities of the delivery device. While in an expanded configuration, the struts 42 may extend radially outwardly from the expandable anchor frame 38.

The threaded region 46 of the central member 44 may extend proximally through the expandable foam body 26 so that a delivery device (not shown) is able to reach the threaded region 46 of the central member 44 in order to rotate the threaded region 46 of the central member 44 relative to the intermediate structure 40. In some cases, the delivery device may instead extend through the expandable foam body 26 in order to engage the threaded region 46 of the central member 44. The intermediate structure 40 may extend proximally through the expandable foam body 26 so that a delivery device (not shown) is able to reach and rotate the threaded region 46 of the central member 44. In some cases, the delivery device may instead extend through the expandable foam body 26 in order to engage the threaded region 46 of the central member 44.

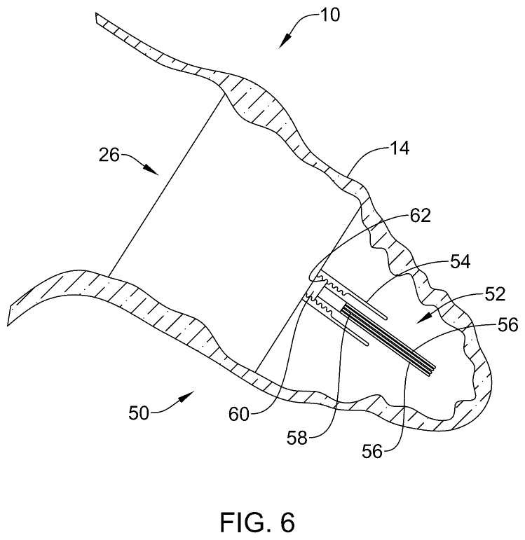

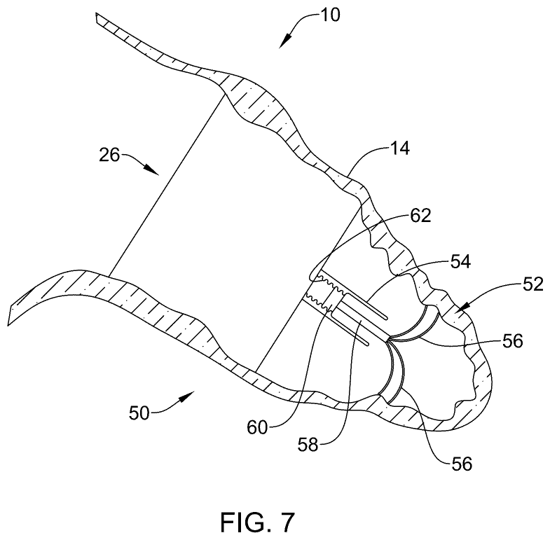

FIG. 6 is a schematic view of an illustrative LAAC device 50 shown disposed within the LAA 10 and FIG. 7 is a schematic view of the illustrative LAAC device 50 of FIG. 6, shown after expansion of an expandable anchor frame. While not shown, at this stage the LAAC device 50 may be coupled with a delivery device. The illustrative LAAC device 50 includes an expandable anchor frame 52 that is expandable from a delivery configuration as shown in FIG. 6 to an expanded anchor configuration as shown for example in FIG. 7. The LAAC device 50 includes an expandable foam body 26 that is expandable from a compressed configuration (not shown) to an expanded configuration as shown in FIGS. 6 and 7. In some cases, the expandable foam body 26 may be adapted to expand from its compressed configuration to its expanded configuration as soon as the expandable foam body 26 is released from a constraining sleeve that holds the expandable foam body 26 in its compressed configuration. In some cases, the expandable foam body 26 may be adapted to regain a remembered shape once no longer constrained, or once the expandable foam body 26 is exposed to increases in temperature and/or moisture (e.g., contacts blood).

In some cases, as shown, the LAAC device 50 also includes an intermediate structure 54. In some cases, the intermediate structure 54 may play a role in moving the expandable anchor frame 52 between its delivery configuration to its expanded anchor configuration. In some cases, the intermediate structure 54 may extend through the expandable foam body 26 such that a delivery device (not shown) may be used to engage the intermediate structure 54 in a manner that allows actuation of the expandable anchor frame 52. The expandable anchor frame 52 may include a plurality of struts 56 that extend distally from a central member 58. The struts 56 may be made of a shape memory material such as nitinol. In some cases, the central member 58 includes a threaded region 60 that is adapted to threadedly engage a threaded region 62 of the intermediate structure 54. Relative rotation of the threaded region 60 relative to the intermediate structure 54 may cause the threaded region 60 of the central member 58 to move distally relative to the intermediate structure 54. As a result, the struts 56 are no longer constrained by the intermediate structure 54, but are able to move in a radially outwardly direction, as seen for example in FIG. 7. In FIG. 7, the expandable anchor frame 52 has moved into its expanded configuration in which the struts 56 are engaging the wall 14 of the LAA 10. The expandable anchor frame 38 may include two, three, four, five, or six struts 42. A maximum number of struts 56 may be determined by overall compressibility of the expandable anchor frame 52 and the capabilities of the delivery device. While in an expanded configuration, the struts 56 may extend radially outwardly from the expandable anchor frame 52.

The threaded region 60 of the central member 58 may extend proximally through the expandable foam body 26 so that a delivery device (not shown) is able to reach the threaded region 60 of the central member 58 in order to rotate the threaded region 60 of the central member 58 relative to the intermediate structure 54. In some cases, the delivery device may instead extend through the expandable foam body 26 in order to engage the threaded region 60 of the central member 58. The intermediate structure 54 may extend proximally through the expandable foam body 26 so that a delivery device (not shown) is able to reach and rotate the threaded region 60 of the central member 58. In some cases, the delivery device may instead extend through the expandable foam body 26 in order to engage the threaded region 60 of the central member 58.

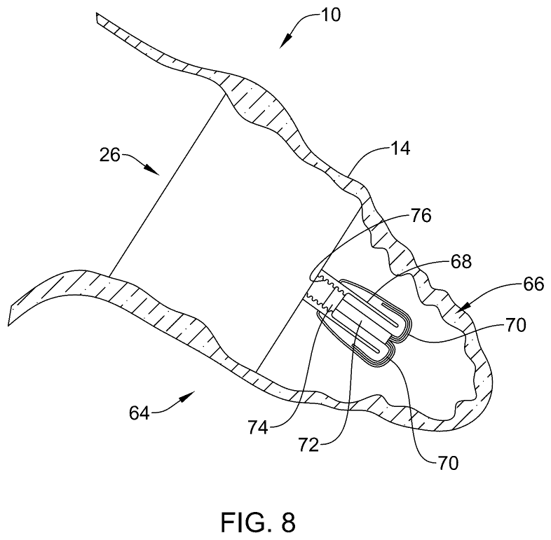

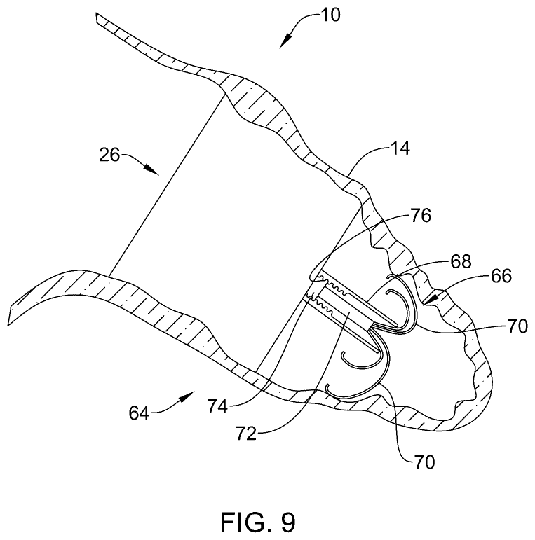

FIG. 8 is a schematic view of an illustrative LAAC device 64 shown disposed within the LAA 10 and FIG. 9 is a schematic view of the illustrative LAAC device 64 of FIG. 8, shown after expansion of an expandable anchor frame. While not shown, at this stage the LAAC device 64 may be coupled with a delivery device. The illustrative LAAC device 64 includes an expandable anchor frame 66 that is expandable from a delivery configuration as shown in FIG. 8 to an expanded anchor configuration as shown for example in FIG. 9. The LAAC device 66 includes an expandable foam body 26 that is expandable from a compressed configuration (not shown) to an expanded configuration as shown in FIGS. 8 and 9. In some cases, the expandable foam body 26 may be adapted to expand from its compressed configuration to its expanded configuration as soon as the expandable foam body 26 is released from a constraining sleeve that holds the expandable foam body 26 in its compressed configuration. In some cases, the expandable foam body 26 may be adapted to regain a remembered shape once no longer constrained, or once the expandable foam body 26 is exposed to increases in temperature and/or moisture (e.g., contacts blood).

In some cases, as shown, the LAAC device 64 also includes an intermediate structure 68. In some cases, the intermediate structure 68 may play a role in moving the expandable anchor frame 66 between its delivery configuration to its expanded anchor configuration. In some cases, the intermediate structure 68 may extend through the expandable foam body 26 such that a delivery device (not shown) may be used to engage the intermediate structure 68 in a manner that allows actuation of the expandable anchor frame 66. The expandable anchor frame 66 may include a plurality of struts 70 that extend distally from a central member 72. The struts 70 may be made of a shape memory material such as nitinol. In some cases, the central member 72 includes a threaded region 74 that is adapted to threadedly engage a threaded region 76 of the intermediate structure 68. Relative rotation of the threaded region 74 relative to the intermediate structure 68 may cause the threaded region 74 of the central member 72 to move distally relative to the intermediate structure 68. As a result, the struts 70, which in their collapsed configuration shown in FIG. 8 are disposed outside of the intermediate structure 68, are caused to move radially outwardly as the threaded region 74 (and the central member 72) moves proximally, thereby gaining their expanded configuration as shown in FIG. 9. In FIG. 9, the expandable anchor frame 66 has moved into its expanded configuration in which the struts 70 are engaging the wall 14 of the LAA 10. The expandable anchor frame 66 may include two, three, four, five, or six struts 70. A maximum number of struts 70 may be determined by overall compressibility of the expandable anchor frame 66 and the capabilities of the delivery device. While in an expanded configuration, the struts 70 may extend radially outwardly from the expandable anchor frame 66.

The threaded region 76 of the central member 72 may extend proximally through the expandable foam body 26 so that a delivery device (not shown) is able to reach the threaded region 74 of the central member 72 in order to rotate the threaded region 74 of the central member 72 relative to the intermediate structure 68. In some cases, the delivery device may instead extend through the expandable foam body 26 in order to engage the threaded region 74 of the central member 72. The intermediate structure 68 may extend proximally through the expandable foam body 26 so that a delivery device (not shown) is able to reach and rotate the threaded region 74 of the central member 72. In some cases, the delivery device may instead extend through the expandable foam body 26 in order to engage the threaded region 74 of the central member 72.

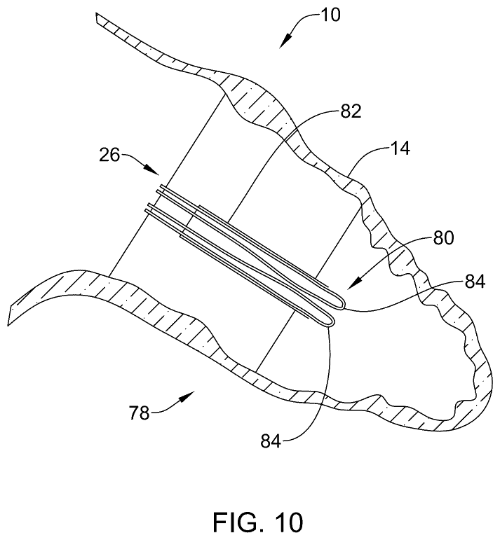

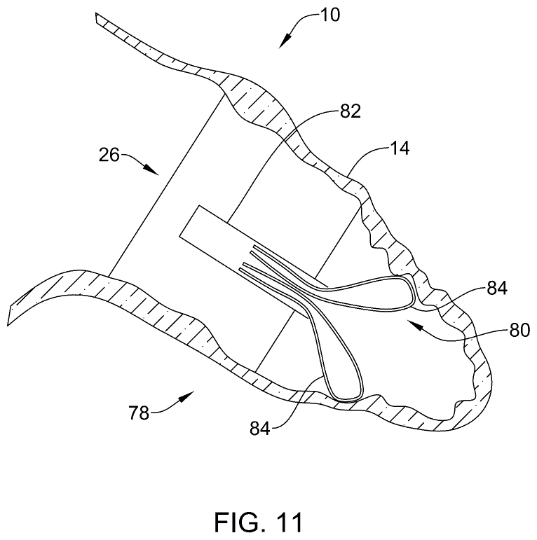

FIG. 10 is a schematic view of an illustrative LAAC device 78 shown disposed within the LAA 10 and FIG. 11 is a schematic view of the illustrative LAAC device 78 of FIG. 10, shown after expansion of an expandable anchor frame. While not shown, at this stage the LAAC device 78 may be coupled with a delivery device. The illustrative LAAC device 78 includes an expandable anchor frame 80 that is expandable from a delivery configuration as shown in FIG. 10 to an expanded anchor configuration as shown for example in FIG. 11. The LAAC device 78 includes an expandable foam body 26 that is expandable from a compressed configuration (not shown) to an expanded configuration as shown in FIGS. 10 and 11. In some cases, the expandable foam body 26 may be adapted to expand from its compressed configuration to its expanded configuration as soon as the expandable foam body 26 is released from a constraining sleeve that holds the expandable foam body 26 in its compressed configuration. In some cases, the expandable foam body 26 may be adapted to regain a remembered shape once no longer constrained, or once the expandable foam body 26 is exposed to increases in temperature and/or moisture (e.g., contacts blood).

In some cases, as shown, the LAAC device 78 also includes a channel 82 that axially extends at least partway through the expandable foam body 26. The expandable anchor frame 80 includes a plurality of struts 84 that extend through the channel 82 such that proximal ends 86 of the struts 84 extend proximally of the channel 82 so that a delivery device (not shown) may be used to translate each of the struts 84 relative to the channel 82. The struts 84 may be made of a shape memory material such as nitinol. In some cases, the struts 84 may be advanced distally through the channel 82 at the same time. In some cases, the struts 84 may be biased to a remembered shape in which the struts 84 extend radially outwardly from the channel 82 when not constrained by the channel 82. In some cases, each of the struts 84 may be individually translatable within the channel 82, such that each of the struts 84 may be advanced an appropriate distance to allow that strut 84 to move radially outwardly as each strut 84 extends out of the channel 82 and contact the wall 14 of the LAA 10 in order to help secure the LAAC device 78 in position. In some cases, each of the struts 84, once positioned, may be locked into position within the channel 82. The expandable anchor 80 may include two, three, four, five, or six struts 84. A maximum number of struts 84 may be determined by overall compressibility of the expandable anchor frame 38 and the capabilities of the delivery device. While in an expanded configuration, the struts 84 may extend radially outwardly from the expandable anchor frame 80.

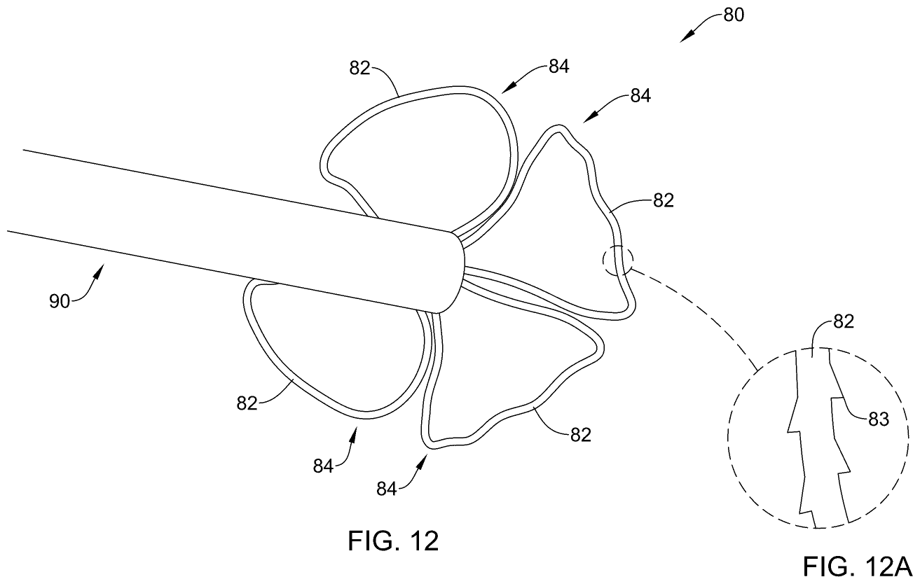

FIG. 12 is a perspective view of the expandable anchor frame 80 disposed within an example delivery device 90. In some cases, the delivery device 90 may extend into and/or through the channel 82 (FIGS. 10 and 11) to deliver and expand the expandable anchor frame 80. In some cases, as shown, the expandable anchor frame 80 may include a total of four struts 82 that are doubled over to form a three-dimensional shape 84 that is adapted to extend radially outwardly and engage the wall 14 of the LAA 10. In some cases, and as seen in FIG. 12A, each of the struts 82 may include micro-teeth 83 that help to engage tissue. In some cases, inclusion of the micro-teeth 83 help the struts 82 to anchor against the wall 14 of the LAA 10. In some cases, the micro-teeth 83 effectively increase a coefficient of friction of the struts 82, which can help the struts 82 remain in place against the wall 14 of the LAA 10, rather than sliding relative to the wall 14 of the LAA 10.

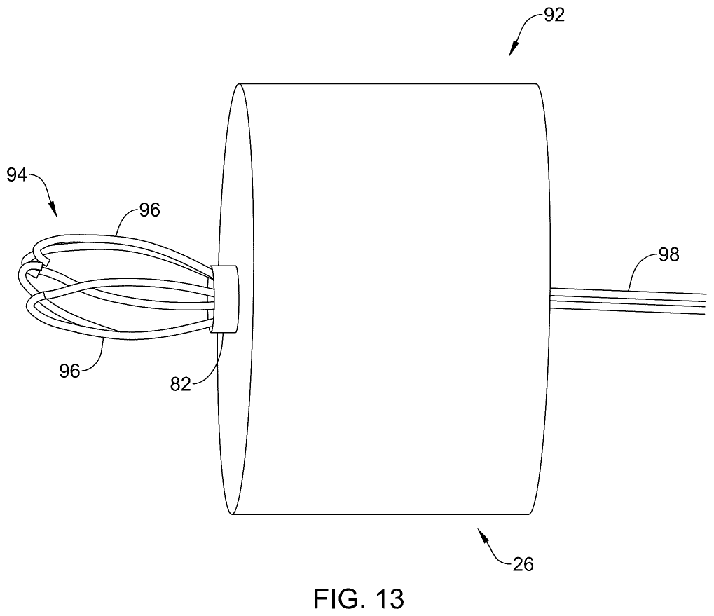

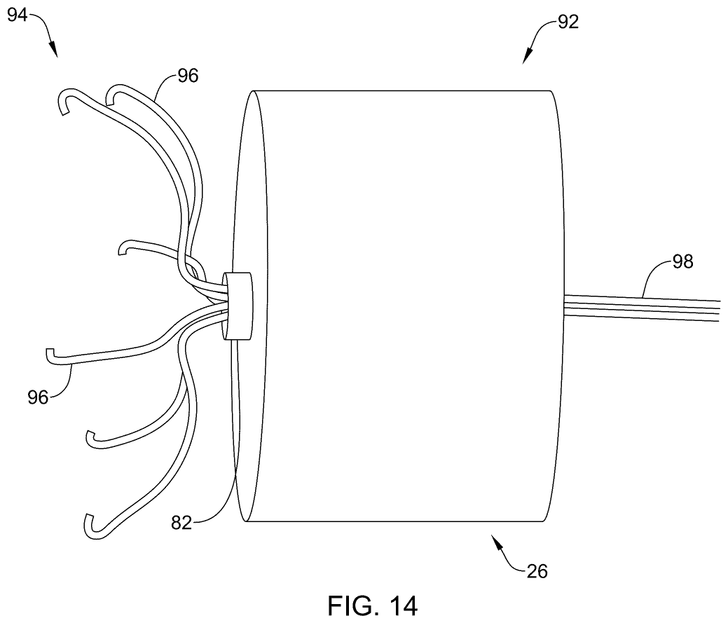

FIG. 13 is a schematic view of an illustrative LAAC device 92 and FIG. 14 is a schematic view of the illustrative LAAC device 92 of FIG. 13, shown after expansion of an expandable anchor frame. The illustrative LAAC device 92 includes an expandable anchor frame 94 that is expandable from a delivery configuration as shown in FIG. 13 to an expanded anchor configuration as shown for example in FIG. 14. The LAAC device 92 includes an expandable foam body 26 that is expandable from a compressed configuration (not shown) to an expanded configuration as shown in FIGS. 13 and 14. In some cases, the expandable foam body 26 may be adapted to expand from its compressed configuration to its expanded configuration as soon as the expandable foam body 26 is released from a constraining sleeve that holds the expandable foam body 26 in its compressed configuration. In some cases, the expandable foam body 26 may be adapted to regain a remembered shape once no longer constrained, or once the expandable foam body 26 is exposed to increases in temperature and/or moisture (e.g., contacts blood).

In some cases, as shown, the LAAC device 92 also includes a channel 82 that axially extends at least partway through the expandable foam body 26. The expandable anchor frame 94 includes a plurality of struts 96 that extend proximally through the channel 82 such that the struts 96 engage with, or extend through, a delivery device 98. The struts 96 may be made of a shape memory material such as nitinol that provides the expandable anchor frame 94 with a remembered shape. When the expandable anchor frame 94 is constrained by extending through the channel 82, the struts 96 are temporarily deformed from the remembered shape of the expandable anchor frame 94. As the expandable anchor frame 94 is no longer constrained by the channel 82 (as seen in FIG. 14), the struts 96 will move in order to re-attain the remembered shape of the expandable anchor frame 94. In some cases, the LAAC device 92 may be considered as a passive device, in which the physician implanting the LAAC device 92 does not have control over the final shape and size of the expandable anchor frame 94. Rather, the final shape and size of the expandable anchor frame 94 may be determined when the expandable anchor frame 94 is processed to achieve the remembered shape of the expandable anchor frame 94. The expandable anchor frame 94 may include two, three, four, five, or six struts 96. A maximum number of struts 96 may be determined by overall compressibility of the expandable anchor frame 94 and the capabilities of the delivery device. While in an expanded configuration, the struts 96 may extend radially outwardly from the expandable anchor frame 94.

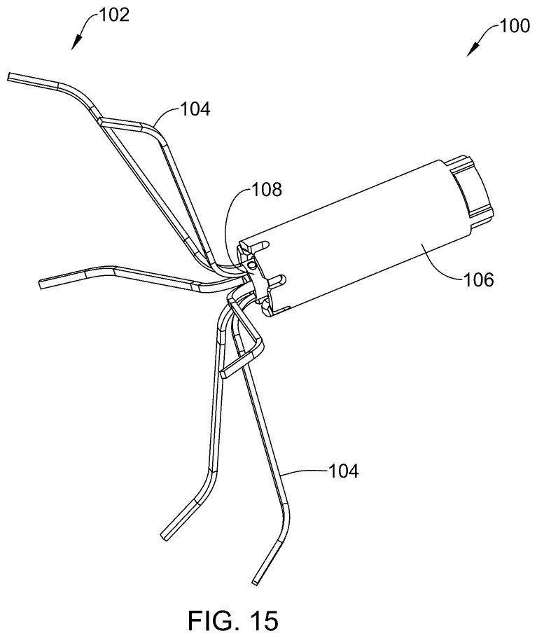

FIG. 15 is a perspective view of a portion of an illustrative LAAC device 100. The illustrative LAAC device 100 includes an expandable anchor frame 102 that includes a plurality of struts 104. The LAAC device 100 may include an expandable foam body (not shown) that is coupled with the expandable anchor frame 102. The expandable anchor frame 102 may include two, three, four, five, or six struts 104. A maximum number of struts 104 may be determined by overall compressibility of the expandable anchor frame 102 and the capabilities of the delivery device. While in an expanded configuration, the struts 104 may extend radially outwardly from the expandable anchor frame 102. The expandable anchor frame 102 is shown in its expanded configuration. A sleeve 106, which in some cases may be part of a delivery device, has been withdrawn proximally in order to allow the expandable anchor frame 102 to expand into its expanded configuration. The expandable anchor frame 102 may be secured to an intermediate structure 108 that is adapted to translate relative to the sleeve 106. The intermediate structure 108, which may be adapted to engage a delivery device (not shown), may be adapted to translate distally relative to the sleeve 106 (or the sleeve 106 may translate proximally relative to the intermediate structure 108) in order to cause the expandable anchor frame 102 to move into its expanded configuration, as shown. The intermediate structure 108 may be adapted to translate proximally relatively to the sleeve 106 (or the sleeve 106 may translate distally relative to the intermediate structure 108) in order to cause the expandable anchor frame 102 to withdraw into the sleeve 106 and hence return to its collapsed or delivery configuration.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D, L-lactide, poly(beta-hydroxybutyrate), polygluconate including poly-D-gluconate, poly-L-gluconate, poly-D, L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha, omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding a patient's left atrial appendage (LAA), the LAAC device comprising:
an expandable anchor frame adapted to expand from a delivery configuration to an expanded anchor configuration; and
an expandable foam body disposed relative to the expandable anchor frame, the expandable foam body adapted to expand from a compressed configuration to an expanded configuration;
wherein expansion of the expandable anchor frame is temporally offset from expansion of the expandable foam body.

2. The LAAC device of claim 1, wherein the expandable anchor frame is adapted to expand before the expandable foam body expands.

3. The LAAC device of claim 1, wherein the expandable foam body is adapted to expand before the expandable anchor frame expands.

4. The LAAC device of claim 1, wherein the expandable anchor frame is adapted to move between the delivery configuration and the expanded anchor configuration by translating the expandable anchor frame relative to the expandable foam body.

5. The LAAC device of claim 1, further comprising a channel extending axially through the expandable foam body.

6. The LAAC device of claim 5, wherein the expandable anchor frame comprises a plurality of individually actuatable struts slidingly disposed within the channel.

7. The LAAC device of claim 6, wherein each of the plurality of individually actuatable struts are securable relative to the LAAC device.

8. The LAAC device of claim 1, further comprising an intermediate structure joining the expandable anchor frame to the expandable foam body.

9. The LAAC device of claim 8, wherein the expandable anchor frame is adapted to move between the delivery configuration and the expanded anchor configuration by rotating the expandable anchor frame relative to the intermediate structure.

10. The LAAC device of claim 8, wherein the expandable frame comprises a plurality of struts extending between a distal hub threadedly engaged with the intermediate structure and a proximal hub secured to the intermediate structure.

11. The LAAC device of claim 10, wherein the expandable anchor frame is adapted to move between the delivery configuration and the expanded anchor configuration by rotating the intermediate structure relative to the distal hub.

12. A left atrial appendage closure (LAAC) device adapted for occluding a patient's left atrial appendage (LAA), the LAAC device comprising:
an expandable anchor frame adapted to expand from a delivery configuration to an expanded anchor configuration; and
an expandable foam body disposed relative to the expandable anchor frame, the expandable foam body adapted to expand from a compressed configuration to an expanded configuration before the expandable anchor frame is expanded.

13. The LAAC device of claim 12, wherein the expandable anchor frame is adapted to translate relative to the expandable foam body.

14. The LAAC device of claim 12, further comprising an intermediate structure joining the expandable anchor frame to the expandable foam body.

15. The LAAC device of claim 14, wherein the expandable anchor frame is adapted to move between the delivery configuration and the expanded anchor configuration by rotating the expandable anchor frame relative to the intermediate structure.

16. The LAAC device of claim 14, wherein the expandable frame comprises a plurality of struts extending between a distal hub threadedly engaged with the intermediate structure and a proximal hub secured to the intermediate structure.

17. The LAAC device of claim 16, wherein the expandable anchor frame is adapted to move between the delivery configuration and the expanded anchor configuration by rotating the intermediate structure relative to the distal hub.

18. A left atrial appendage closure (LAAC) device adapted for occluding a patient's left atrial appendage (LAA), the LAAC device comprising:
an expandable anchor frame adapted to expand from a delivery configuration to an expanded anchor configuration;
an expandable foam body disposed relative to the expandable anchor frame, the expandable foam body adapted to expand from a compressed configuration to an expanded configuration; and
an intermediate structure joining the expandable anchor frame to the expandable foam body.

19. The LAAC device of claim 18, wherein the expandable anchor frame is adapted to move between the delivery configuration and the expanded anchor configuration by rotating the expandable anchor frame relative to the intermediate structure.

20. The LAAC device of claim 18, wherein the expandable frame comprises a plurality of struts extending between a distal hub threadedly engaged with the intermediate structure and a proximal hub secured to the intermediate structure, and the expandable anchor frame is adapted to move between the delivery configuration and the expanded anchor configuration by rotating the intermediate structure relative to the distal hub.
